(19) United States
(12) Patent Application Publication
(10) Pub. No.: US 2024/0180487 A1
(43) Pub. Date: Jun. 6, 2024

(54) ELECTRONIC DEVICE

(71) Applicant: Advanced Semiconductor Engineering, Inc., Kaohsiung (TW)

(72) Inventor: Chun-Kai CHANG, Kaohsiung (TW)

(73) Assignee: Advanced Semiconductor Engineering, Inc., Kaohsiung (TW)

(21) Appl. No.: 18/073,522

(22) Filed: Dec. 1, 2022

(51) Int. Cl. A61B 5/00

(52) U.S. Cl. CPC A61B 5/6801; A61B 2562/164; A61B 2562/166

(57) ABSTRACT

The present disclosure provides an electronic device. The electronic device includes a carrier having a component side and a sensing side opposite to the component side. The sensing side has a thinned portion. The electronic device also includes a first sensing element disposed over the sensing side and a second sensing element disposed over the sensing side. The first sensing element and the second sensing element are arranged along a primary direction of the electronic device. The thinned portion is between the first sensing element and the second sensing element and is configured to provide adjustment to a relative position between the first sensing element and the second sensing element.

### BACKGROUND

### 1. Technical Field

The present disclosure relates to an electronic device.

### 2. Description of the Related Art

Electronic wearable devices are becoming increasingly functional. For example, components or packages (such as system in packages (SiPs)) may be integrated into a flexible molding compound of an electronic wearable device to perform functions, such as obtaining information or signals reflecting physical activity and/or health, capturing pictures, connecting to the Internet, etc.

The Young's modulus of the components are greater than that of the flexible molding compound. Thus, stress due to transformation of the flexible molding compound tends to concentrate on the edges or boundaries of the components. The flexible molding compound might deteriorate (crack or break) when the electronic wearable device is repeatedly attached to and detached from a person's body. It is preferable that the electronic wearable device can inhibit deterioration of the flexible molding compound and can have a curved shape so as to conform to a curved surface of the body or be curved according to the movement of the body.

### SUMMARY

In some arrangements, an electronic device includes a carrier having a component side and a sensing side opposite to the component side. The sensing side has a thinned portion. The electronic device also includes a first sensing element disposed over the sensing side and a second sensing element disposed over the sensing side. The first sensing element and the second sensing element are arranged along a primary direction of the electronic device. The thinned portion is between the first sensing element and the second sensing element and is configured to provide adjustment to a relative position between the first sensing element and the second sensing element.

In some arrangements, an electronic device includes a carrier having a first surface and a second surface opposite to the first surface. The carrier has a thinned portion. The electronic device includes a first sensing element disposed over the first surface of the carrier and adjacent to the thinned portion, and an electronic component disposed over the second surface of the carrier. The first sensing element and the electronic component are at least partially overlapped along a direction substantially perpendicular to the second surface of the carrier.

In some arrangements, an electronic device includes a carrier having a component side and a sensing side opposite to the component side. The sensing side has a thinned portion. The electronic device includes a first sensing element disposed over the sensing side and a second sensing element disposed over the sensing side. The thinned portion is between the first sensing element and the second sensing element and is configured to allow the first sensing element and the second sensing element to respectively move toward two directions. An angle between the two directions is greater than zero.

### BRIEF DESCRIPTION OF THE DRAWINGS

Aspects of some arrangements of the present disclosure are best understood from the following detailed description when read with the accompanying figures. It is noted that various structures may not be drawn to scale, and dimensions of the various structures may be arbitrarily increased or reduced for clarity of discussion.

FIG. 1A illustrates a cross-sectional view of an electronic device in accordance with some arrangements of the present disclosure.

FIG. 1B illustrates a cross-sectional view of an electronic device in accordance with some arrangements of the present disclosure.

FIG. 1C illustrates a cross-sectional view of a part of an electronic device in accordance with some arrangements of the present disclosure.

FIG. 1D illustrates a cross-sectional view of a part of an electronic device in accordance with some arrangements of the present disclosure.

FIG. 1E illustrates a cross-sectional view of an electronic device in accordance with some arrangements of the present disclosure.

FIG. 1F illustrates a cross-sectional view of an electronic device in accordance with some arrangements of the present disclosure.

FIG. 2A illustrates a top view of an electronic device in accordance with some arrangements of the present disclosure.

FIG. 2B illustrates a bottom view of an electronic device in accordance with some arrangements of the present disclosure.

FIG. 2C illustrates a perspective view of an electronic device in accordance with some arrangements of the present disclosure.

FIG. 2C′ provides an alternative view of an electronic device of FIG. 2C in accordance with some arrangements of the present disclosure.

FIG. 2D illustrates a conceptual view showing a state in which a user wears an electronic device in accordance with some arrangements of the present disclosure.

FIG. 3A illustrates a cross-sectional view of a part of an electronic device in accordance with some arrangements of the present disclosure.

FIG. 3B illustrates a cross-sectional view of a part of an electronic device in accordance with some arrangements of the present disclosure.

FIG. 3C illustrates a cross-sectional view of a part of an electronic device in accordance with some arrangements of the present disclosure.

FIG. 3D illustrates a cross-sectional view of a part of an electronic device in accordance with some arrangements of the present disclosure.

FIG. 3E illustrates a cross-sectional view of a part of an electronic device in accordance with some arrangements of the present disclosure.

FIG. 4A illustrates a bottom view of a part of an electronic device in accordance with some arrangements of the present disclosure.

FIG. 4B illustrates a bottom view of a part of an electronic device in accordance with some arrangements of the present disclosure.

FIG. 4C illustrates a bottom view of a part of an electronic device in accordance with some arrangements of the present disclosure.

FIG. 4D illustrates a bottom view of a part of an electronic device in accordance with some arrangements of the present disclosure.

FIG. 4E illustrates a bottom view of a part of an electronic device in accordance with some arrangements of the present disclosure.

FIG. 4F illustrates a bottom view of a part of an electronic device in accordance with some arrangements of the present disclosure.

FIG. 5 illustrates a cross-sectional view of an electronic device in accordance with some arrangements of the present disclosure.

FIG. 6A, FIG. 6B, FIG. 6C, and FIG. 6D illustrate top views and cross-sectional views in one or more stages of a method of manufacturing an electronic device in accordance with an arrangement of the present disclosure.

### DETAILED DESCRIPTION

The following disclosure provides for many different arrangements, or examples, for implementing different features of the provided subject matter. Specific examples of components and arrangements are described as follows to explain certain aspects of the present disclosure. These are, of course, merely examples and are not intended to be limiting. For example, the formation of a first feature over or on a second feature in the description that follows may include arrangements in which the first and second features are formed or disposed in direct contact, and may also include arrangements in which additional features may be formed or disposed between the first and second features, such that the first and second features may not be in direct contact. In addition, the present disclosure may repeat reference numerals and/or letters in the various examples. This repetition is for the purpose of simplicity and clarity and does not in itself dictate a relationship between the various arrangements and/or configurations discussed.

Spatial descriptions, such as “above,” “below,” “up,” “left,” “right,” “down,” “top,” “bottom,” “vertical,” “horizontal,” “side,” “higher,” “lower,” “upper,” “over,” “under,” and so forth, are indicated with respect to the orientation shown in the figures unless otherwise specified. It should be understood that the spatial descriptions used herein are for purposes of illustration only, and that practical implementations of the structures described herein can be spatially arranged in any orientation or manner, provided that the merits of the arrangements of this disclosure are not deviated from by such arrangement.

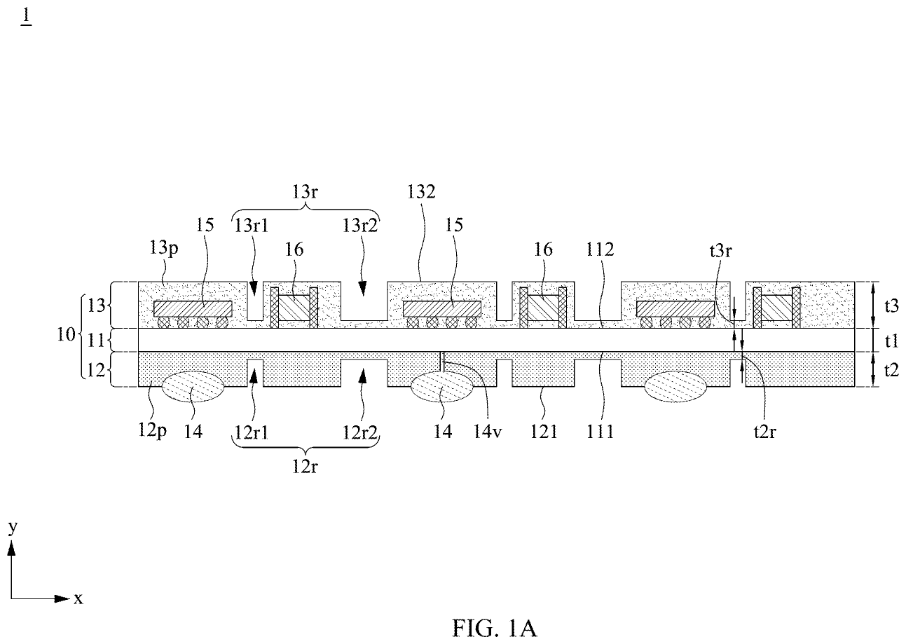

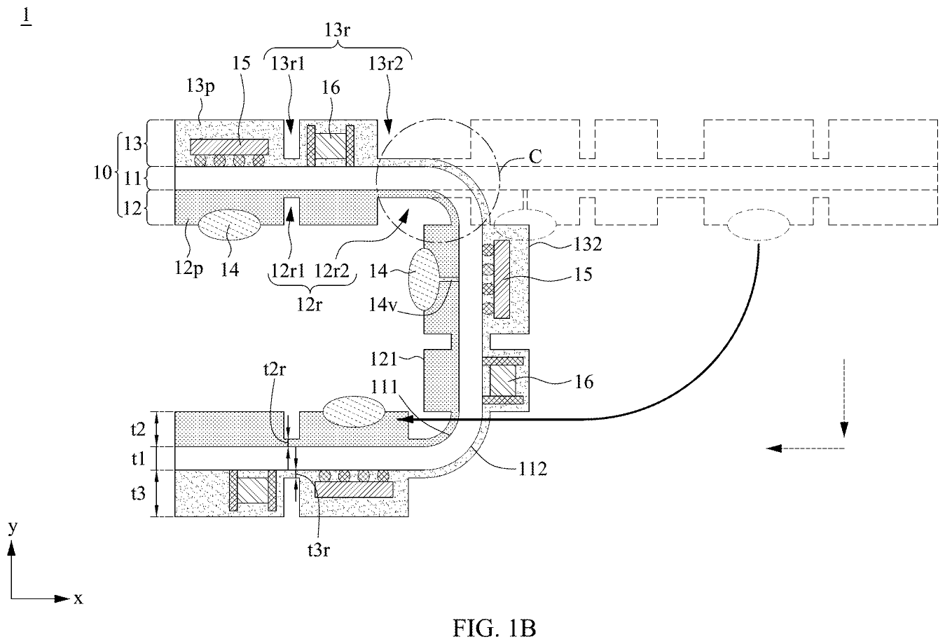

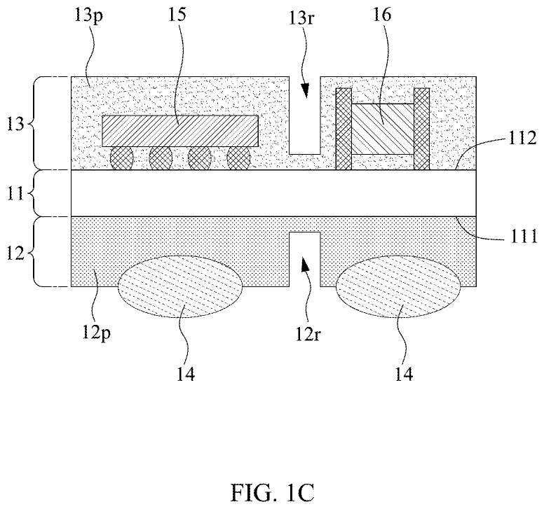

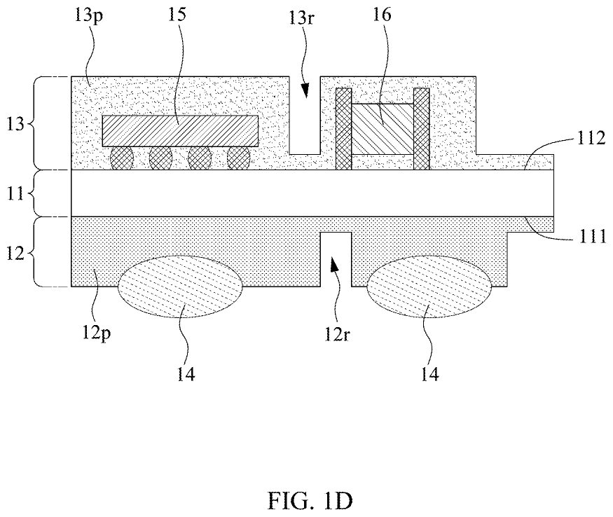

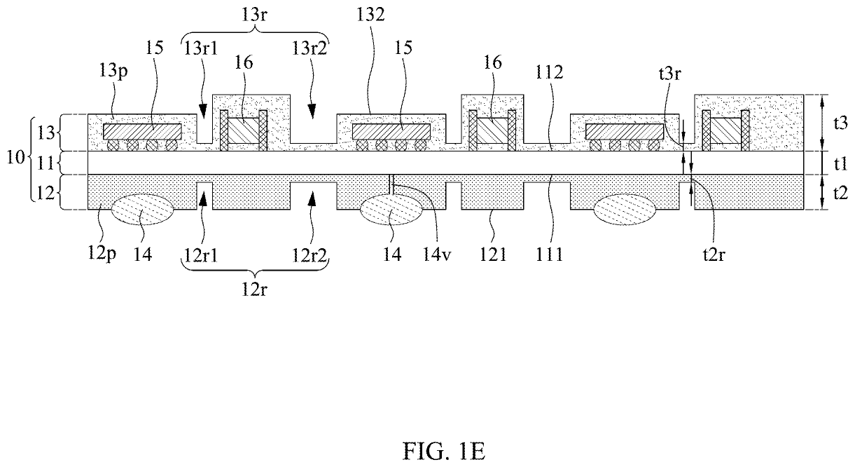

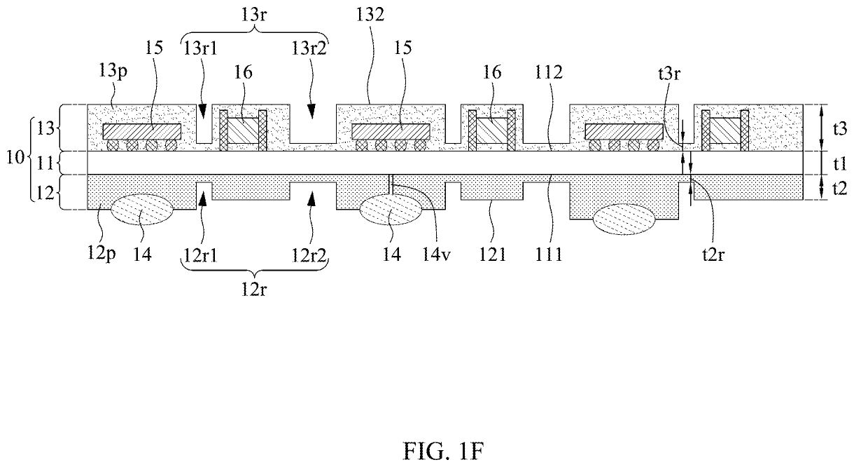

FIG. 1A illustrates a cross-sectional view of an electronic device 1 in accordance with some arrangements of the present disclosure. In some arrangements, the electronic device 1 may include a wearable device, such as a smartwatch, a smart band, or another smart wearable device. Configuration or application of the electronic device 1 in the figures is for illustrative purposes only, and not intended to limit the present disclosure.

In some arrangements, the electronic device 1 may be a piece of equipment that detects an external signal by using various detection devices (such as sensors). In some arrangements, the electronic device 1 may perform data communication with a base station or a terminal device (such as a mobile phone) in a wireless communications manner, such as via radio frequency identification technology or short-range wireless communications technology. In some arrangements, the electronic device 1 may be used in combination with a detection device (such as a sensor), an electronic device (such as a signal processing device) and/or other corresponding external devices for further processing acquired signals.

Referring to FIG. 1A, the electronic device 1 may include a carrier 10, one or more sensing elements 14, one or more electronic components 15, and one or more electronic components 16.

The carrier 10 may include a plurality of sublayers. For example, the carrier 10 in FIG. 1A has three sublayers 11, 12, and 13. However, the present disclosure is not limited thereto. The number of sublayers of the carrier 10 may be adjusted based on design requirements. For example, in some other arrangements, the sublayers 11 and 12 may equal a monolithic layer or a monolithic carrier, and the sublayer 13 may be a protection layer disposed over or on the monolithic layer to cover the electronic components 15 and 16. In some other arrangements, the sublayers 11, 12, and 13 may be a monolithic layer or a monolithic carrier. For example, the carrier 10 may include a one-piece molded layer.

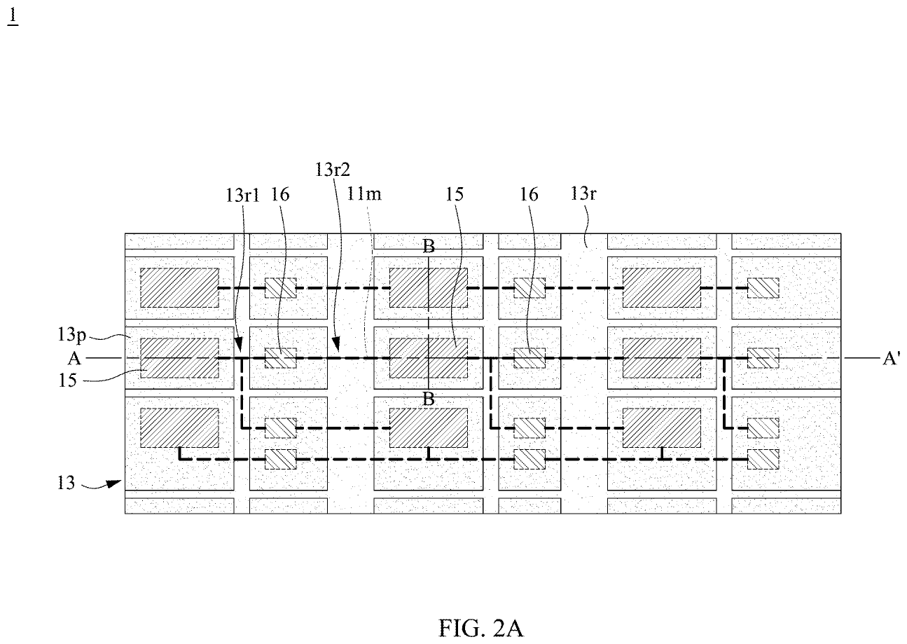

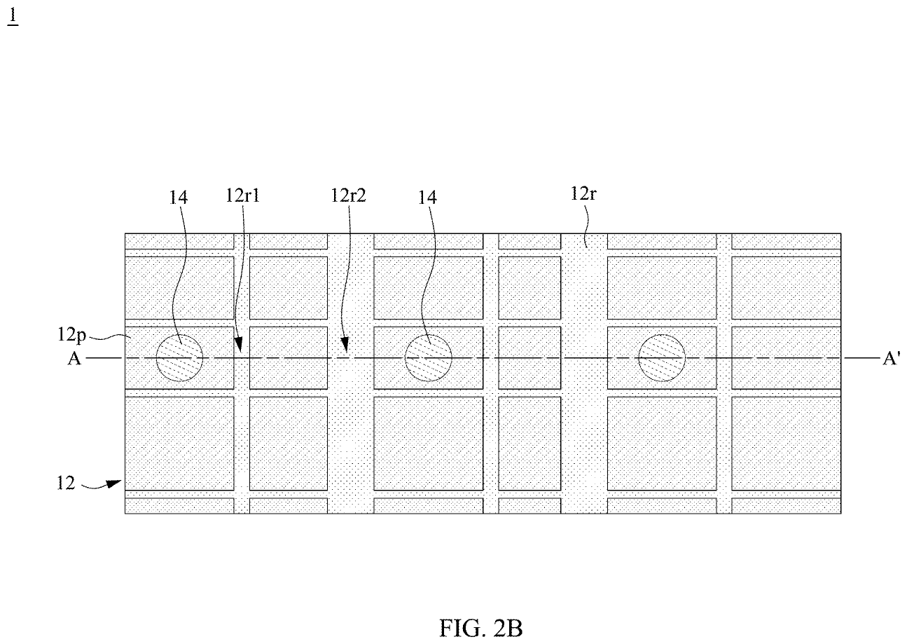

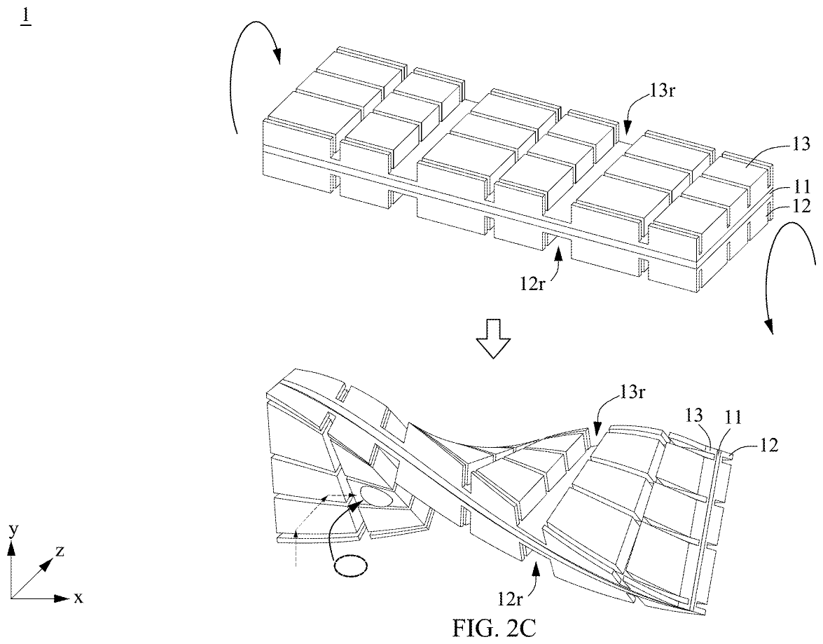

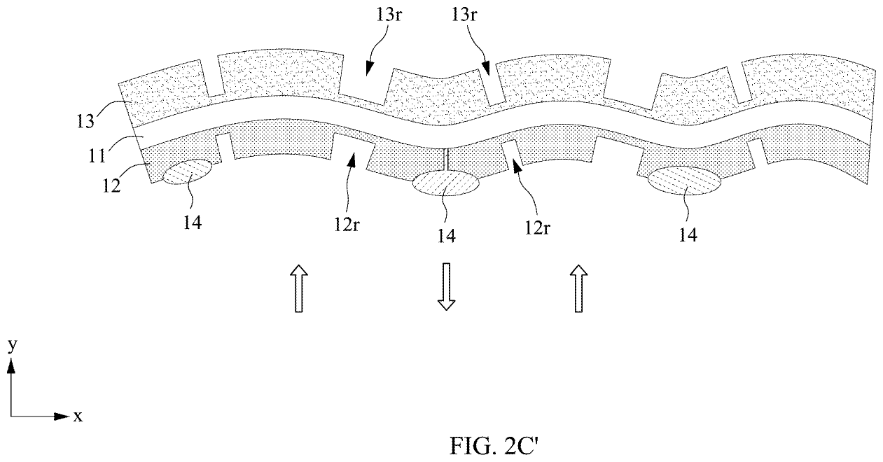

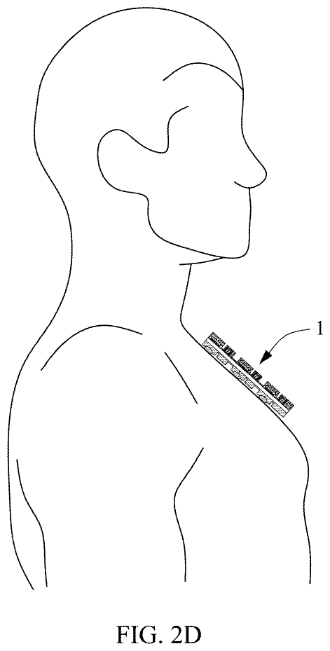

The sublayer 11 may include a supporting layer for structurally supporting the electronic components 15 and 16. The sublayer 11 may include a copper plate, a flexible printed circuit board, or a flexible substrate. The sublayer 11 may include an interconnection structure (such as the interconnection structure 11m in FIG. 2A), such as a redistribution layer (RDL), a circuit layer, a conductive trace, a conductive via, etc. The interconnection structure may provide signal paths for the components (such as the sensing elements 14, and the electronic components 15 and 16) electrically connected with the sublayer 11. In some arrangements, the sublayer 11 may include a surface 111 and a surface 112 opposite to the surface 111. The surface 112 may include a component side and the surface 111 may include a sensing side. In some arrangements, the sublayer 11 may have a dimension (such as thickness, width, and/or cross section) t1 ranging from about 35 μm to about 65 μm, such as about 50 μm. In some arrangements, the dimension t1 may be the distance between the surface 111 and the surface 112.

The sublayer 12 may be disposed over or on the surface 111 of the sublayer 11. The sublayer 13 may be disposed over or on the surface 112 of the sublayer 11. The sublayer 11 may be disposed between the sublayers 12 and 13. In some arrangements, the sublayer 12 may be configured to face an object to be detected. For example, when the electronic device 1 is worn by a user, the sublayer 12 may face the user's skin.

In some arrangements, the sublayer 12 may include a dielectric material. The dielectric material may include, for example, but is not limited to, phosphoric anhydride (PA), a polyimide (PI), a polybenzoxazole (PBO), Borophosphosilicate Glass (BPSG), Undoped Silicate Glass (USG), silicon oxide, silicon nitride, silicon oxynitride, any combination of two or more thereof, or the like. In some arrangements, the sublayer 12 may include rubber, silicon, sponge, or other suitable materials such as an elastic material, a soft material, or a flexible material. In some embodiments, the sublayer 12 may include a liquid silicone rubber (LSR). In some embodiments, the sublayer 12 may be configured to be adjustable. For example, when the electronic device 1 is worn by a user, the sublayer 12 may flexibly adjust its shape to conform to a body part of the user. The sublayer 12 may be soft and flexible enough for a user to wear comfortably for an extended time.

The sublayer 12 may include recessed portions or recessed regions 12r which are recessed from the top surface 121 of the sublayer 12 facing away from the sublayer 11. The recessed portions 12r may define thinned portions of the sublayer 12 that have smaller dimensions (such as thicknesses, widths, and/or cross sections) with respect to protruding portions 12p of the sublayer 12. The protruding portions 12p may be separated from one another by the recessed portions 12r. The protruding portions 12p and the recessed portions 12r may be arranged in an alternating way.

For example, the sublayer 12 may have a dimension (such as thickness, width, and/or cross section) t2 ranging from about 85 μm to about 115 μm, such as about 100 μm. In some arrangements, the dimension t2 may be the distance between the surface 111 and the top surface 121 of the sublayer 12. In some arrangements, the dimension t2 may be greater than or equal to twice of the dimension t1 of the sublayer 11.

The recessed portions 12r of the sublayer 12 may have dimensions (such as thicknesses, widths, and/or cross sections) t2r ranging from about 15 μm to about 45 μm, such as about 30 μm. In some arrangements, the dimension t2r may be the distance between the surface 111 and the bottoms (not labelled in the figures) of the recessed portions 12r of the sublayer 12. In some arrangements, the dimension t2r may be less than or equal to a half of the dimension t2. In some arrangements, the dimension t2r may be less than or equal to one third of the dimension t2.

The sensing elements 14 may be disposed over or on the sublayer 12. For example, the sensing elements 14 may be disposed over or on the top surface 121 of the sublayer 12. The sensing elements 14 may each be at least partially surrounded, embedded, or covered by the sublayer 12. In some other arrangements, one or more of the sensing elements 14 may be entirely surrounded, embedded, or covered by the sublayer 12.

The sensing elements 14 may be disposed over or on the portions (such as protruding portions 12p) other than the recessed portions 12r. For example, the sensing elements 14 may be spaced apart from the recessed portions 12r. The sensing elements 14 may be separated from one another by the recessed portions 12r.

In some arrangements, the recessed portions 12r may be relatively more flexible or bendable than the protruding portions 12p. Therefore, the recessed portions 12r may also be referred to as flexible or bendable portions. The protruding portions 12p may also be referred to as rigid portions. In some arrangements, the relative positions of the protruding portions 12p may be adjusted by changing the structure or the shape of the recessed portions 12r, such as by bending, twisting, stretching, squishing the recessed portions 12r. Therefore, the relative positions of the sensing elements 14 may be adjusted by changing the structure or the shape of the recessed portions 12r.

The recessed portions 12r may be configured to provide adjustment to a relative position between two of the sensing elements 14 in at least two directions or dimensions.

The recessed portions 12r may be configured to allow the sensing elements 14 to move toward different directions or dimensions. For example, the recessed portions 12r may be configured to allow two of the sensing elements 14 to respectively or individually move toward two directions and an angle between the two directions may be greater than zero.

For example, the recessed portions 12r may be configured to displace two of the sensing elements 14 along different Cartesian axes. For example, the recessed portions 12r may be configured to displace one of the sensing elements 14 along a first Cartesian axis and to displace another one of the sensing elements 14 along a second Cartesian axis different from the first Cartesian axis.

The recessed portions 12r may be configured to change a relative elevation of the sensing elements 14. The recessed portions 12r may be configured to allow two of the sensing elements 14 to face toward each other. The recessed portions 12r may be configured to decrease a distance between two of the sensing elements 14.

The sensing elements 14 may each include a conductive pad or a sensing pad. For example, the sensing elements 14 may each include an electrode, a thermistor, a pressure sensor, a proximity sensor, a motion sensor, an acoustic sensor, a smell sensor, a particle sensor, a humidity sensor, an optical transmitter, an optical receiver, an optical transceiver, or a combination thereof.

In some arrangements, the sensing elements 14 may each be configured to detect or collect one or more signals or pieces of information external to the electronic device 1. For example, the sensing elements 14 may each be configured to detect light, sound, temperature, air pressure, smell, particle, humidity, or other environmental variables. For example, when the electronic device 1 is worn by a user, the sensing elements 14 may be configured to detect or collect one or more signals (e.g., biosignals) or pieces of information associated with the user.

The shape, location, and number of the sensing elements 14 in FIG. 1A are for illustrative purposes only, and not intended to limit the present disclosure. In some other arrangements, the sensing elements 14 may be disposed over any one or more of the protruding portions 12p. In some other arrangements, two or more sensing elements 14 may be disposed over or on the same one of the protruding portions 12p. In some arrangements, the protruding portions 12p may be configured to provide adjustment to a relative position between the electronic device 1 and a user wearing the electronic device 1. In some arrangements, the protruding portions 12p may be configured for pressing along a direction substantially perpendicular to the top surface 121 of the sublayer 12.

In some arrangements, one or more of the sensing elements 14 may be electrically connected to the interconnection structure of the sublayer 11 through a conductive element 14v. The conductive element 14v may penetrate the sublayer 12 and may include a conductive via, a conductive pillar, a conductive wire, a conductive trace, etc. The shape, location, and number of the conductive element 14v in FIG. 1A are for illustrative purposes only, and not intended to limit the present disclosure.

In some arrangements, the sensing elements 14 may be electrically connected to the electronic component 15 through the sublayer 11. In some arrangements, the biosignals detected by the sensing elements 14 may be further processed by the electronic components 15 to determine a biological parameter of the user, such as a pulse travel time (PTT), an electroencephalogram (EEG), electrocardiogram (ECG), electromyogram (EMG), electrooculogram (EOG), galvanic skin response (GSR), sweat composition, pH, heart rate variability (HRV), or other biologically-relevant information associated with the user.

In some arrangements, the conductive element 14v may be configured to provide vertical electrical connections between one of the sensing elements 14 and the closest one of the electronic components 15 to decrease the signal loss. However, in some other arrangements, one of the electronic components 15 may be configured to process the biosignals from two or more of the sensing elements 14.

In some arrangements, the sublayer 13 may include an encapsulant. The encapsulant may include, for example, but is not limited to, an epoxy resin having fillers, a molding compound (e.g., an epoxy molding compound or other molding compound), a phenolic compound or material, a material with a silicone dispersed therein, any combination of two or more thereof, or the like. In some arrangements, the encapsulant may include an opaque material. In some arrangements, the opaque material may be an opaque epoxy (e.g., a black epoxy) or other opaque resin or polymer. In some arrangements, the encapsulant may include a light transmissive material. In some arrangements, the light transmissive material may be a clear epoxy or other light transmissive epoxy or other resin or polymer.

In some arrangements, the sublayer 12 and the sublayer 13 may have the same material. In some arrangements, the sublayer 12 and the sublayer 13 may have different materials. For example, the sublayer 12 may include a PI layer and the sublayer 13 may include a molding compound layer.

The electronic components 15 and 16 may be disposed over or on the surface 112 of the sublayer 11 and covered by the sublayer 13. In some arrangements, the electronic components 15 and 16 may each be electrically connected to one or more other devices (if any) and to the carrier 10, and the electrical connection may be attained by way of flip-chip, wire-bond techniques, metal to metal bonding (such as Cu to Cu bonding), or hybrid bonding.

In some arrangements, the electronic components 15 may each include an active device or an active component. The electronic components 15 may each be or include circuits or circuit elements that rely on an external power supply to control or modify electrical signals. In some arrangements, the electronic components 16 may each include a passive device or a passive component, such as resistors, capacitors, inductors, or a combination thereof.

In some arrangements, the electronic components 15 may each include a processing device, a storage device, or a transmission device. For example, the electronic components 15 may each be configured to process (e.g., analysis, modify, synthesize, convert to a digital signal, and amplify, etc.), to store, and/or to transmit the signals (such as biosignals) detected by the sensing elements 14.

The positions, functions, and number of electronic components in the electronic device 1 are not intended to limit the present disclosure. For example, there may be any number of electronic components in the electronic device 1 due to design requirements.

The sublayer 13 may include recessed portions 13r which are recessed from the top surface 132 of the sublayer 13 facing away from the sublayer 11. The recessed portions 13r may define thinned portions of the sublayer 13 that have smaller dimensions (such as thicknesses, widths, and/or cross sections) with respect to protruding portions 13p of the sublayer 13. The protruding portions 13p may be separated from one another by the recessed portions 13r. The protruding portions 13p and the recessed portions 13r may be arranged in an alternating way.

For example, the sublayer 13 may have a dimension (such as thickness, width, and/or cross section) t3 ranging from about 1000 μm to about 1200 μm, such as about 1100 μm. In some arrangements, the dimension t3 may be the distance between the surface 112 and the top surface 132 of the sublayer 13. In some arrangements, the dimension t3 may be greater than or equal to twenty times of the dimension t1 of the sublayer 11. In some arrangements, the dimension t3 may be greater than or equal to ten times of the dimension t2 of the sublayer 12.

The recessed portions 13r of the sublayer 13 may have dimensions (such as thicknesses, widths, and/or cross sections) t3r ranging from about 85 μm to about 115 μm, such as about 100 μm. In some arrangements, the dimension t3r may be the distance between the surface 112 and the bottoms (not labelled in the figures) of the recessed portions 13r of the sublayer 13. In some arrangements, the dimension t3r may be less than or equal to one tenth of the dimension t3. In some arrangements, the dimension t3r may be greater than or equal to three times of the dimension t2r of the recessed portions 12r of the sublayer 12.

The electronic components 15 and 16 may each be covered by one of the protruding portions 13p. In some other arrangements, two or more of the electronic components 15 and 16 may be covered by the same one of the protruding portions 13p.

The electronic components 15 and 16 may each be spaced apart from the recessed portions 13r. The electronic components 15 and 16 may be separated from one another by the recessed portions 13r.

In some arrangements, the recessed portions 13r may be relatively more flexible or bendable than the protruding portions 13p. Therefore, the recessed portions 13r may also be referred to as flexible or bendable portions. The protruding portions 13p may also be referred to as rigid portions. In some arrangements, the relative positions of the protruding portions 13p may be adjusted by changing the structure or the shape of the recessed portions 13r, such as by bending, twisting, stretching, squishing the recessed portions 13r. Therefore, the relative positions of the electronic components 15 and 16 may be adjusted by changing the structure or the shape of the recessed portions 13r.

The recessed portions 13r may be configured to provide adjustment to a relative position between the electronic components 15 and 16 in at least two directions or dimensions.

The recessed portions 13r may be configured to allow the electronic components 15 and 16 to move toward different directions or dimensions. For example, the recessed portions 13r may be configured to allow the electronic components 15 and 16 to respectively or individually move toward two directions and an angle between the two directions may be greater than zero.

For example, the recessed portions 13r may be configured to displace the electronic components 15 and 16 along different Cartesian axes. For example, the recessed portions 13r may be configured to displace the electronic component 15 along a first Cartesian axis and to displace the electronic component 16 along a second Cartesian axis different from the first Cartesian axis.

The recessed portions 13r may be configured to change a relative elevation of the electronic components 15 and 16. The recessed portions 13r may be configured to allow two of the electronic components 15 and 16 to face toward each other. The recessed portions 13r may be configured to decrease a distance between two of the electronic components 15 and 16.

In some arrangements, in a direction substantially perpendicular to the surface 112 and/or the surface 111 of the sublayer 11, the recessed portions 12r of the sublayer 12 and the recessed portions 13r of the sublayer 13 may be at least partially overlapped. For example, as shown in FIG. 1A, the recessed portions 12r of the sublayer 12 may each be aligned with one of the recessed portions 13r of the sublayer 13. For example, the recessed portion 12rl may be aligned with the recessed portion 13rl and the recessed portion 12r2 may be aligned with the recessed portion 13r2.

For example, as shown in FIG. 1A, the protruding portions 12p of the sublayer 12 may each be aligned with one of the protruding portions 13p of the sublayer 13.

In some arrangements, the electronic components 15 may be larger than the electronic components 16. Therefore, the protruding portions 13p covering the electronic components 15 may be larger than the protruding portions 13p covering the electronic components 16. Therefore, the protruding portions 13p covering the electronic components 15 may be more rigid the protruding portions 13p covering the electronic components 16. The structural strength of the protruding portions 13p covering the electronic components 15 may be greater than the structural strength of the protruding portions 13p covering the electronic components 16.

In some arrangements, in a direction substantially perpendicular to the surface 112 and/or the surface 111 of the sublayer 11, the electronic components 15 and the sensing elements 14 may be at least partially overlapped. For example, the sensing elements 14 may be disposed according to the locations of the electronic components 15. For example, the sensing elements 14 may be disposed over or on the protruding portions 12p right below the electronic components 15. The protruding portions 12p right below the electronic components may provide structural support for the sensing elements 14 and avoid bending the sensing elements 14.

For example, in a direction substantially perpendicular to the surface 112 and/or the surface 111 of the sublayer 11, the electronic components 16 and the sensing elements 14 may not be overlapped.

FIG. 1B illustrates a cross-sectional view of the electronic device 1 in accordance with some arrangements of the present disclosure.

The electronic device 1 may be bended greater than or equal to 90 degrees. For example, as marked by the dotted circles C, the recessed region 12r may have a curved surface. The protruding portions 12p connected to the recessed region 12r may be closer to each other. The recessed region 13r may have a curved surface. The protruding portions 13p connected to the recessed region 13r may be farther from each other.

The portions of the surface 111 of the sublayer 11 over which the protruding portions 12p are disposed are angled at about 90 degrees. When the electronic device 1 is bended greater than 90 degrees, the angle defined between the two portions of the surface 111 of the sublayer 11 may be less than 90 degrees.

The recessed portions 12r may be configured to provide adjustment to a relative position between two of the sensing elements 14 in two components (such as the x-component and the y-component) of a vector.

According to some arrangements of the present disclosure, the carrier 10 has the thinned portions (such as the recessed portions 12r and the recessed portions 13r) to increase the flexibility, such as to increase the moving directions and to increase the bending angles. The recessed portions 12r may be configured to be compressed or absorb energy of compressive stress. The recessed portions 13r may be configured to be stretched or absorb energy of tensile stress. For example, the electronic device 1 is omni-direction bendable. For example, the electronic device 1 may be twisted as shown in FIG. 2C. For example, the electronic device 1 can be bended greater than or equal to 270 degrees. In some arrangements, the electronic device 1 may be relatively more resistant to stress, impact, twisting or other physical or structural changes. For example, the electronic device 1 may be resilient, such that, after being squeezed or pressed, it can return to its original state without deteriorating (such as cracking or breaking). In addition, since some parts of the carrier 10 are thinner, the manufacturing cost of the electronic device 1 may be reduced and the electronic device 1 can be more compact and lightweight.

Furthermore, when the electronic device 1 is worn by a user, the carrier 10 can flexibly adjust its shape to conform to a body part of the user. Therefore, the relative positions of the sensing elements 14 can be adjusted and the sensing elements 14 can adhere to a body part of the user. For example, as shown in FIG. 2D, the electronic device 1 may be used to adhere to a chest of the user and function as an electrode patch. The sensibility of the sensing elements 14 can be enhanced and the sensing efficiency can be increased.

FIG. 1C illustrates a cross-sectional view of a part of an electronic device in accordance with some arrangements of the present disclosure. The part of the electronic device in FIG. 1C is similar to a part of the electronic device 1 in FIG. 1A except for the differences described as follows.

In some arrangements, the sensing elements 14 may be separated from each other by the recessed portion 12r. The electronic component 15 and electronic component 16 may be separated from each other by the recessed portion 13r. In a direction substantially perpendicular to the surface 112 and/or the surface 111 of the sublayer 11, the electronic component 16 and the sensing element 14 may be at least partially overlapped. For example, the sensing element 14 may be disposed over or on the protruding portion 12p right below the electronic component 16.

In some arrangements, the dimension (such as the surface area) of the sensing element 14 overlapped with the electronic component 16 may be smaller than the dimension (such as the surface area) of the sensing element 14 overlapped with the electronic component 15.

FIG. 1D illustrates a cross-sectional view of a part of an electronic device in accordance with some arrangements of the present disclosure. The part of the electronic device in FIG. 1D is similar to a part of the electronic device 1 in FIG. 1A except for the differences described as follows.

In some arrangements, the sensing elements 14 may be separated from each other by the recessed portion 12r. The electronic component 15 and electronic component 16 may be separated from each other by the recessed portion 13r.

In a direction substantially perpendicular to the surface 112 and/or the surface 111 of the sublayer 11, the recessed portion 12r may be non-overlapped with the recessed portion 13r. In a direction substantially perpendicular to the surface 112 and/or the surface 111 of the sublayer 11, the recessed portion 12r and the recessed portion 13r may be staggered. The recessed portion 12r may be misaligned with the recessed portion 13r. The recessed portion 12r may not be aligned with the recessed portion 13r. The recessed portion 12r may be spaced apart from the recessed portion 13r.

In a direction substantially perpendicular to the surface 112 and/or the surface 111 of the sublayer 11, the recessed portion 12r may be overlapped with the protruding portion 13p. The recessed portion 13r may be overlapped with the protruding portion 12p.

In some arrangements, when the carrier 10 is bended, the protruding portions 12p and 13p may support the sublayer 11 and prevent the interconnection structure (such as the interconnection structure 11m in FIG. 2A) in the sublayer 11 from delamination or being cracked.

FIG. 1E illustrates a cross-sectional view of an electronic device in accordance with some arrangements of the present disclosure. The electronic device in FIG. 1E is similar to the electronic device 1 in FIG. 1A except that the top surface 132 of the sublayer 13 may have different elevations. For example, the protruding portions 13p of the sublayer 13 covering the electronic components 16 may be thicker than the protruding portions 13p of the sublayer 13 covering the electronic components 15.

FIG. 1F illustrates a cross-sectional view of an electronic device in accordance with some arrangements of the present disclosure. The electronic device in FIG. 1F is similar to the electronic device 1 in FIG. 1A except that the top surface 121 of the sublayer 12 may have different elevations. For example, the protruding portions 12p of the sublayer 12 over which the sensing elements 14 are disposed may be thicker than the other protruding portions 12p. In some arrangements, the top surface 132 of the sublayer 13 of the electronic device in FIG. 1F may have different elevations.

FIG. 2A illustrates a top view of the electronic device 1 in accordance with some arrangements of the present disclosure. In some arrangements, the electronic device 1 in FIG. 1A may be the cross-sectional view cut through line AA′ in FIG. 2A.

The recessed portions 13r may have trenches running in two directions, such as in horizontal and vertical directions. The two directions may be orthogonal. The recessed portions 13r may define a mesh pattern. For example, the recessed portions 13r may define a plurality of blocks. The recessed portions 13r may be connected to one another. In some arrangements, the dimensions (such as thicknesses, widths, and/or cross sections) of the trenches may be different. For example, a thicker vertical trench may be disposed next to a thinner vertical trench.

The protruding portions 13p may not be connected to one another. The protruding portions 13p may be isolated from one another. In some arrangements, the surface area of each of the protruding portions 13p may be greater than or equal to about 100 mm². Therefore, each of the protruding portions 13p may be configured to accommodate one electronic component 15 of about 100 mm² or four electronic components 16 of about 25 mm². The surface area and the number of protruding portions 13p are for illustrative purposes only, and not intended to limit the present disclosure.

FIG. 2B illustrates a bottom view of the electronic device 1 in accordance with some arrangements of the present disclosure. In some arrangements, the electronic device 1 in FIG. 1A may be the cross-sectional view cut through line AA′ in FIG. 2B.

The recessed portions 12r may have trenches running in two directions, such as in horizontal and vertical directions. The recessed portions 12r may define a mesh pattern. For example, the recessed portions 12r may define a plurality of blocks. The recessed portions 12r may be connected to one another. In some arrangements, the dimensions (such as thicknesses, widths, and/or cross sections) of the trenches may be different. For example, a thicker vertical trench may be disposed next to a thinner vertical trench.

The protruding portions 12p may not be connected to one another. The protruding portions 12p may be isolated from one another. In some arrangements, the surface area of each of the protruding portions 12p may be greater than or equal to about 100 mm². Therefore, each of the protruding portions 12p may be configured to accommodate one sensing element 14 of about 100 mm² or four sensing elements 14 of about 25 mm². The surface area and the number of protruding portions 12p are for illustrative purposes only, and not intended to limit the present disclosure.

FIG. 2C illustrates a perspective view of the electronic device 1 in accordance with some arrangements of the present disclosure.

As the arrows point out, a part of the carrier 10 may be twisted or rotated. For example, a part of the carrier 10 may be twisted in a clockwise direction and an opposite part of the carrier 10 may be twisted in a counterclockwise direction. The carrier 10 may be twisted such that two portions of the sublayer 13 may face two opposite directions. The recessed portions 12r may be configured to provide adjustment to a relative position between two of the sensing elements 14 in three components (such as the x-component, the y-component, and the z-component) of a vector.

FIG. 2D illustrates a conceptual view showing a state in which a user wears the electronic device 1 in accordance with some arrangements of the present disclosure. The electronic device 1 may be used to adhere to a chest of the user and function as an electrode patch.

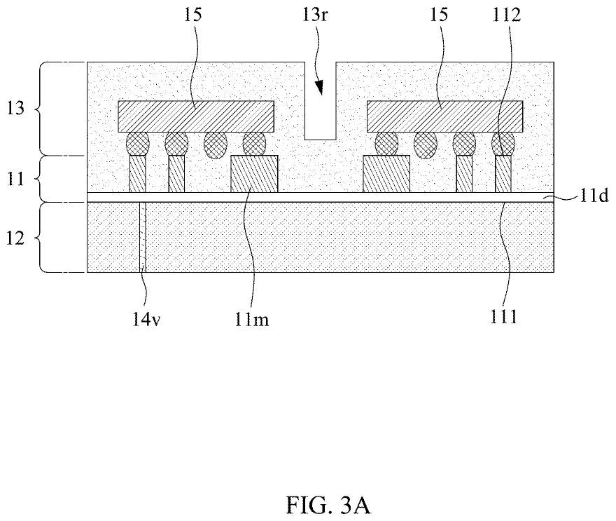

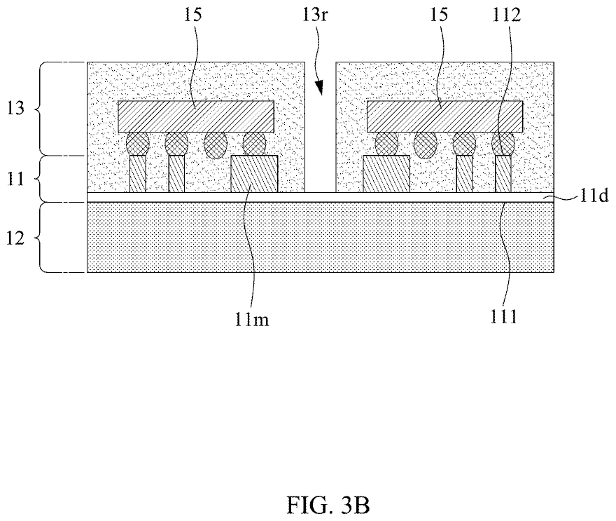

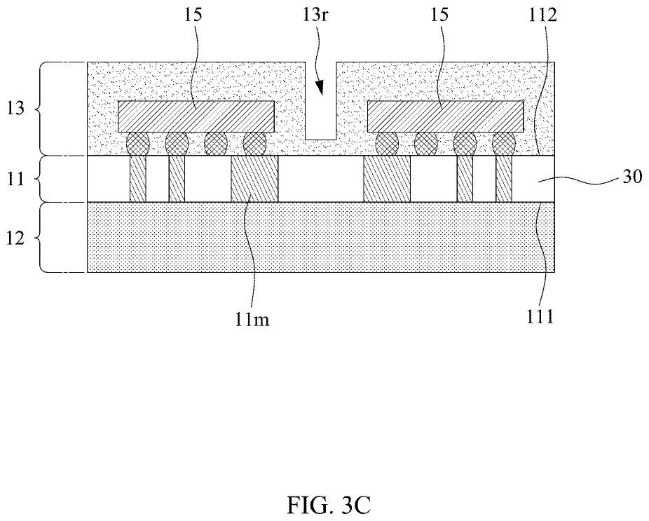

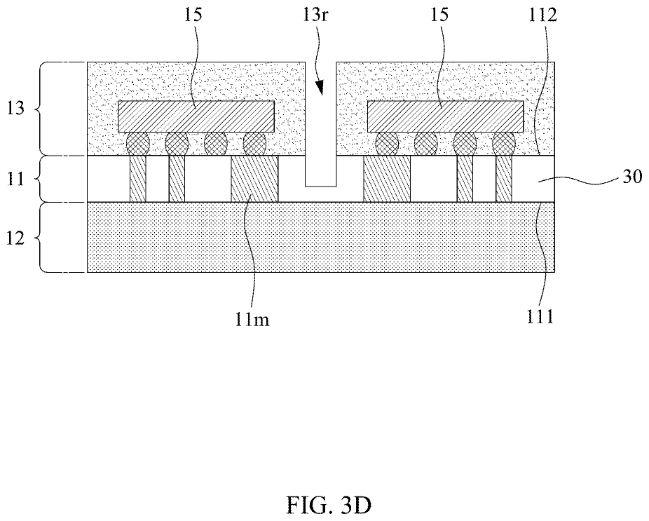

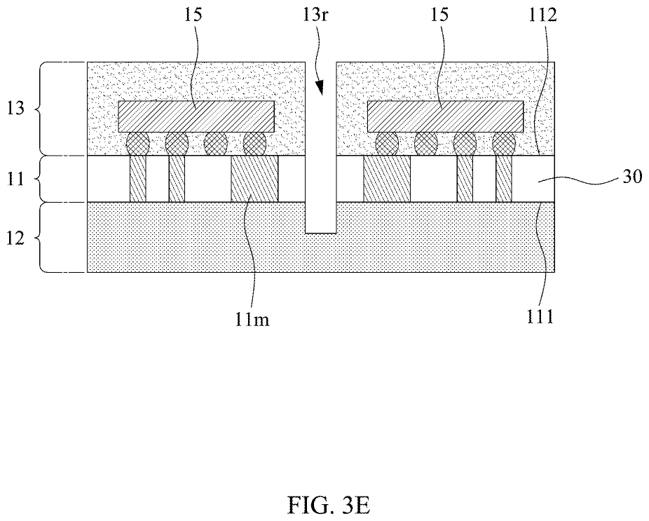

FIGS. 3A-3E illustrate cross-sectional views of a part of an electronic device in accordance with some arrangements of the present disclosure. In some arrangements, the electronic device 1 in FIG. 1A may have the cross-sectional views in FIGS. 3A-3E. The similar or same components are labeled by the same symbols. The differences are described as follows.

Referring to FIGS. 3A and 3B, the parts of the electronic devices in FIGS. 3A and 3B may be the cross-sectional views cut through line BB′ in FIG. 2A. The interconnection structure 11m may include conductive lines or circuits separated from one another. The interconnection structure 11m may be covered by the sublayer 13.

For example, the sublayer 11 may have the surface (or a bottom surface) 111 facing away from the sublayer 12, the surface (or a top surface) 112 opposite to the surface 111, and a lateral surface (not labelled in the figures) extending between the surface 111 and the surface 112. The sublayer 11 may include a dielectric layer 11d and the interconnection structure 11m disposed over the dielectric layer 11d. The sublayer 13 may contact the lateral surface and the top surface of the interconnection structure 11m.

In some arrangements, the recessed portion 13r may not extend into the sublayer 11 as shown in FIG. 3A. For example, the bottom of the recessed portion 13r may not extend into the interconnection structure 11m. For example, the bottom of the recessed portion 13r is disposed at an elevation higher than the top surface of the interconnection structure 11m.

In some arrangements, the recessed portion 13r may extend into the sublayer 11 as shown in FIG. 3B. For example, the bottom of the recessed portion 13r may extend into the interconnection structure 11m. For example, the bottom of the recessed portion 13r is disposed at an elevation lower than the top surface of the interconnection structure 11m.

Referring to FIGS. 3C, 3D, and 3E, the sublayer 11 may include a protection layer 30 covering the interconnection structure 11m. In some arrangements, the protection layer 30 may include a solder resist or a solder mask.

In some arrangements, the recessed portion 13r may not extend into the sublayer 11 as shown in FIG. 3C. For example, the bottom of the recessed portion 13r may not extend into the protection layer 30. In some arrangements, the recessed portion 13r may extend into the sublayer 11 as shown in FIG. 3D. For example, the bottom of the recessed portion 13r may extend into the protection layer 30. In some arrangements, the recessed portion 13r may extend into the sublayer 12 as shown in FIG. 3E.

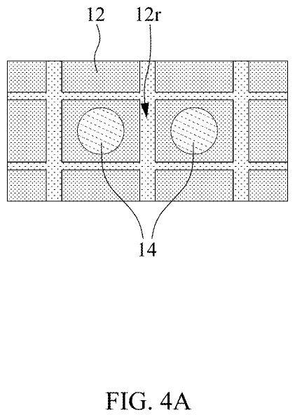

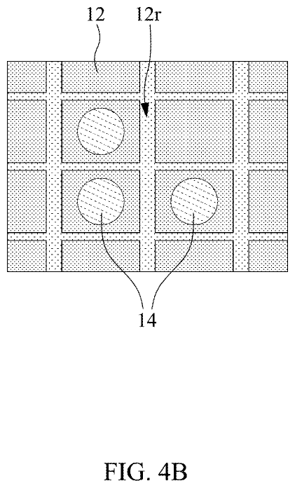

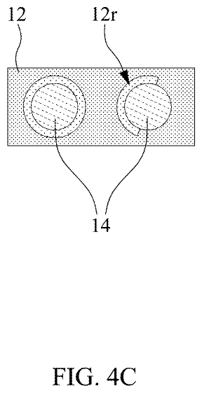

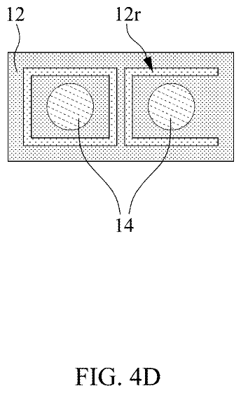

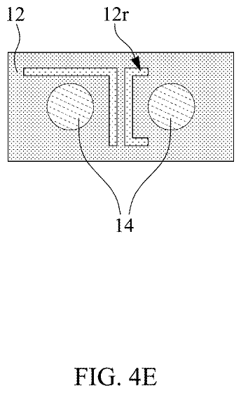

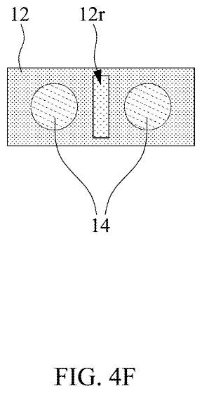

FIGS. 4A-4F illustrate bottom views of a part of an electronic device in accordance with some arrangements of the present disclosure. In some arrangements, the electronic device 1 in FIG. 1A may have the cross-sectional views in FIGS. 4A-4F. The similar or same components are labeled by the same symbols. The differences are described as follows.

Referring to FIGS. 4A and 4B, the sensing elements 14 may be surrounded by the mesh pattern defined by the recessed portion 12r. The sensing elements 14 may be disposed in adjacent blocks. The sensing elements 14 may be arranged along one or more directions. The sensing elements 14 may be arranged along a primary direction and a secondary direction different from the primary direction. The primary direction may include a direction along a first Cartesian axis (such as the x-axis, the y-axis, or the z-axis shown in FIG. 2C.). The secondary direction may include a direction along a second Cartesian axis different from the first Cartesian axis. In some arrangements, the primary direction may include the bending direction of the electronic device 1 when the electronic device 1 is worn by a user. For example, the primary direction may be around the wrist of the user (or a curved surface of the user's skin).

Referring to FIG. 4C, the recessed portion 12r may form along a boundary or a circumference of the sensing element 14. The recessed portion 12r may be connected to the sensing element 14 from a bottom view. For example, the sensing element 14 on the left may have a rounded boundary and the recessed portion 12r may fully encircle the rounded boundary of the sensing element 14. On the other hand, the recessed portion 12r on the right may only encircle half of the section around the rounded boundary of the sensing element 14 on the right.

Referring to FIG. 4D, the recessed portion 12r on the left may have a rectangular shape from a bottom view. The recessed portion 12r on the left may have four linear sections or segments around the sensing element 14 on the left. The sensing element 14 on the left is surrounded by the rectangular shape. The recessed portion 12r on the right may have three linear sections or segments around the sensing element 14 on the right. The rectangular shape on the left may not be connected to the three linear sections or segments on the right.

Referring to FIG. 4E, the recessed portion 12r on the left may have two linear sections around the sensing element 14 on the left. The recessed portion 12r on the right may have three linear sections around the sensing element 14 on the right.

Referring to FIG. 4F, the recessed portion 12r may have a singular linear section between the sensing elements 14. The recessed portion 12r may separate the sensing elements 14. The recessed portion 12r may not have a bending portion. The recessed portion 12r may extend along a direction different from the arranging direction of the sensing elements 14. For example, the sensing elements 14 may be arranged along a horizontal direction and the recessed portion 12r may extend along a vertical direction.

The shape, position, relative location/position, length, and number of the sections of the recessed portions 12r in FIGS. 4A-4F are for illustrative purposes only, and not intended to limit the present disclosure.

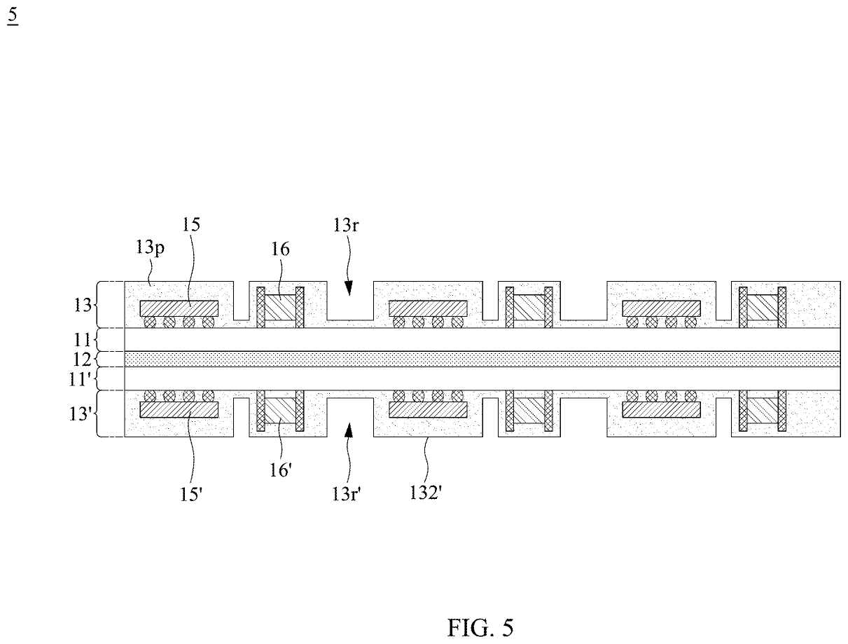

FIG. 5 illustrates a cross-sectional view of an electronic device 5 in accordance with some arrangements of the present disclosure. The electronic device 5 is similar to the electronic device 1 in FIG. 1A except for the differences described as follows.

The electronic device 5 includes sublayers 11, 11′, 12, 13, and 13′. The sublayer 11′ may be similar to the sublayer 11. The sublayer 13′ may be similar to the sublayer 13. The sublayer 12 may be disposed between the sublayers 11 and 11′. The sublayer 11′ may be disposed between the sublayers 12 and 13′. The electronic components 15′ and 16′ may be disposed over or on the sublayer 11′ and covered by the sublayer 13′.

The sublayer 13′ may include recessed portions 13r′ which are recessed from the top surface 132′ of the sublayer 13′ facing away from the sublayer 11′. The electronic components 15′ and 16′ may each be spaced apart from the recessed portions 13r′. The electronic components 15′ and 16′ may be separated from one another by the recessed portions 13r′. The relative positions of the electronic components 15′ and 16′ may be adjusted by changing the structure or the shape of the recessed portions 13r′. In some arrangements, there may be more sublayers in the electronic device 5 to electrically connect more electronic components and to provide more functions.

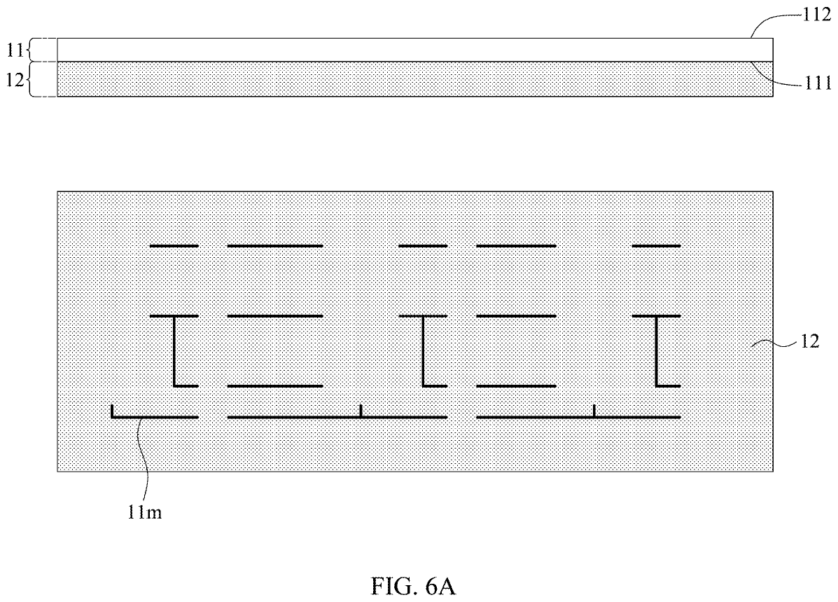

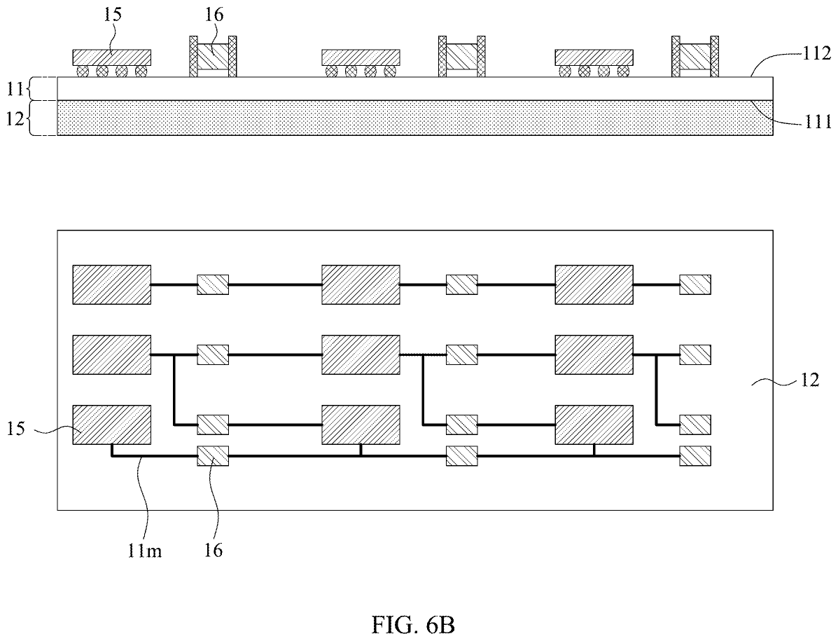

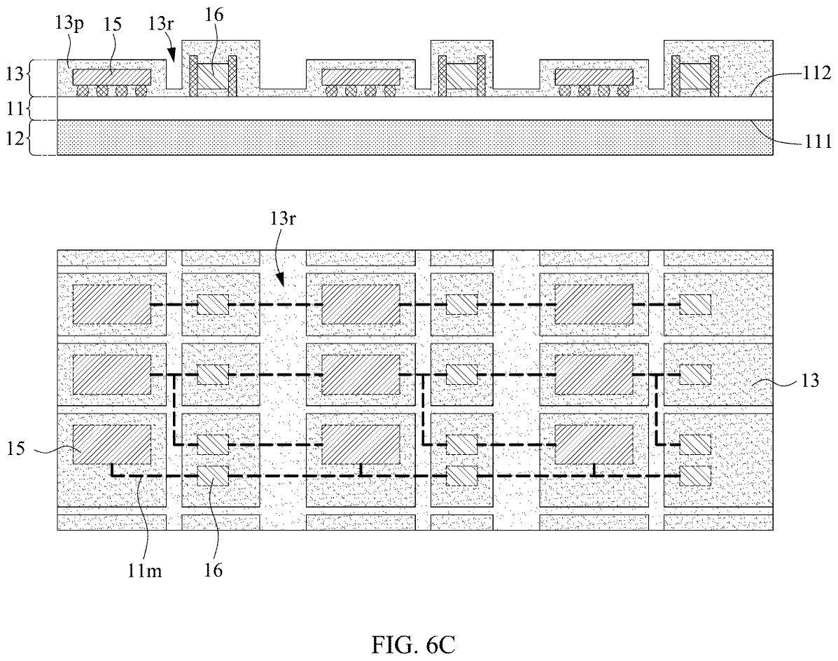

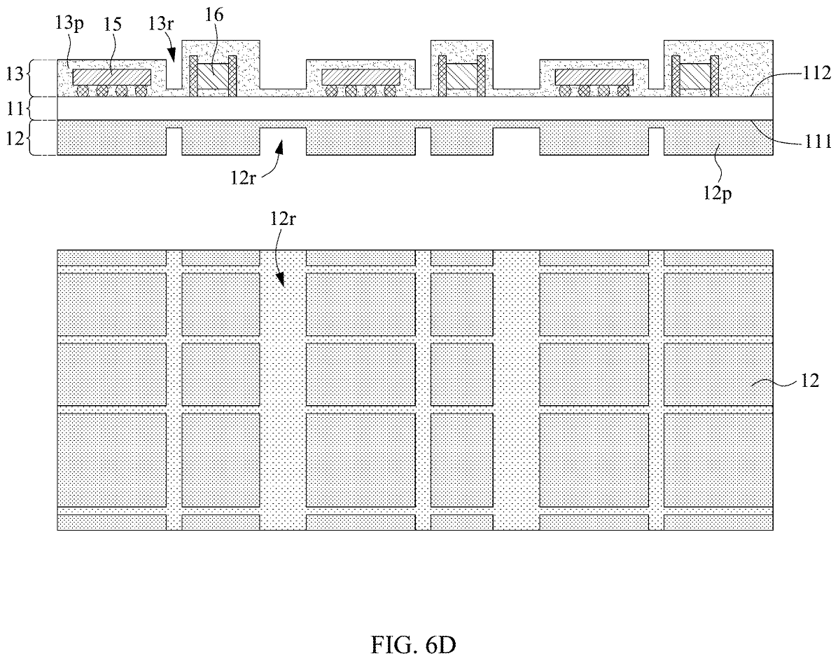

FIG. 6A, FIG. 6B, FIG. 6C, and FIG. 6D illustrate top views and cross-sectional views in one or more stages of a method of manufacturing an electronic device in accordance with an arrangement of the present disclosure. At least some of these figures have been simplified to better understand the aspects of the present disclosure. In some arrangements, the electronic device 1 may be manufactured through the operations described with respect to FIG. 6A, FIG. 6B, FIG. 6C, and FIG. 6D.

Referring to FIG. 6A, the sublayer 12 may be provided. The interconnection structure 11m may be disposed over or on the sublayer 12 and forming the sublayer 11. The pattern or the routing of the interconnection structure 11m may be predetermined based on the locations of the electronic components in the following operations. From a cross-sectional view, the sublayer 11 may include the surface 111 and the surface 112 opposite to the surface 111. In some other arrangements, the sublayer 11 may further include a protection layer 30 covering the interconnection structure 11m, as shown in FIGS. 3C, 3D, and 3E.

Referring to FIG. 6B, the electronic components 15 and 16 may be disposed over or on the sublayer 11 and electrically connected to the interconnection structure 11m.

Referring to FIG. 6C, the sublayer 13 may be disposed over or on the sublayer 11 to cover the electronic components 15 and 16. In some arrangements, the sublayer 13 may be formed by a molding technique, such as transfer molding or compression molding. The sublayer 13 may have the recessed portions 13r and the protruding portions 13p. The electronic components 15 and 16 may be separated from one another by the recessed portions 13r.

In some arrangements, the recessed portions 13r and the protruding portions 13p may be formed by removing parts of the sublayer 13 through, for example, laser cutting technology. In some arrangements, the recessed portions 13r and the protruding portions 13p may be formed along with the transfer molding or compression molding. For example, the shapes of the recessed portions 13r and the protruding portions 13p may be predefined by a casting mold. For example, the recessed portions 13r and the protruding portions 13p may be formed without removing parts of the sublayer 13.

Referring to FIG. 6D, portions of the sublayer 12 may be removed to form the recessed portions 12r and the protruding portions 12p through, for example, laser cutting technology. In some arrangements, the sensing elements 14 in FIG. 1A may be disposed over or on the protruding portions 12p after the removing operations in FIG. 6D. In some other arrangements, the sensing elements 14 in FIG. 1A may be disposed over or on the protruding portions 12p before the removing operations in FIG. 6D.

In some arrangements, a singulation may be performed to separate out individual electronic devices. The singulation may be performed, for example, by using a dicing saw, laser or other appropriate cutting techniques.

As used herein, the singular terms “a,” “an,” and “the” may include a plurality of referents unless the context clearly dictates otherwise.

As used herein, the terms “conductive,” “electrically conductive” and “electrical conductivity” refer to an ability to transport an electric current. Electrically conductive materials typically indicate those materials that exhibit little or no opposition to the flow of an electric current. One measure of electrical conductivity is Siemens per meter (S/m). Typically, an electrically conductive material is one having a conductivity greater than approximately 10⁴ S/m, such as at least 10⁵ S/m or at least 10⁶ S/m. The electrical conductivity of a material can sometimes vary with temperature. Unless otherwise specified, the electrical conductivity of a material is measured at room temperature.

As used herein, the terms “approximately,” “substantially,” “substantial” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, two numerical values can be deemed to be “substantially” the same or equal if a difference between the values is less than or equal to ±10% of an average of the values, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” parallel can refer to a range of angular variation relative to 0° that is less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°. For example, “substantially” perpendicular can refer to a range of angular variation relative to 90° that is less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

While the present disclosure has been described and illustrated with reference to specific arrangements thereof, these descriptions and illustrations do not limit the present disclosure. It should be understood by those skilled in the art that various changes may be made and equivalents may be substituted without departing from the true spirit and scope of the present disclosure as defined by the appended claims. The illustrations may not be necessarily drawn to scale. There may be distinctions between the artistic renditions in the present disclosure and the actual apparatus due to manufacturing processes and tolerances. There may be other arrangements of the present disclosure which are not specifically illustrated. The specification and drawings are to be regarded as illustrative rather than restrictive. Modifications may be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it will be understood that these operations may be combined, sub-divided, or re-ordered to form an equivalent method without departing from the teachings of the present disclosure. Accordingly, unless specifically indicated herein, the order and grouping of the operations are not limitations of the present disclosure.

What is claimed is:

1. An electronic device, comprising:
a carrier having a component side and a sensing side opposite to the component side, the sensing side having a thinned portion;
a first sensing element disposed over the sensing side; and
a second sensing element disposed over the sensing side, wherein the first sensing element and the second sensing element are arranged along a primary direction of the electronic device,
wherein the thinned portion is between the first sensing element and the second sensing element and is configured to provide adjustment to a relative position between the first sensing element and the second sensing element.

2. The electronic device of claim 1, wherein the sensing side comprises a sublayer, and the thinned portion is recessed from a first surface of the sublayer, wherein the first surface is distal from the sensing side.

3. The electronic device of claim 2, further comprising:
a first electronic component disposed over the component side of the carrier; and
a first protection layer covering the first electronic component,
wherein the first protection layer has a recessed portion adjacent to the first electronic component.

4. The electronic device of claim 3, wherein the recessed portion and the thinned portion are staggered along a direction substantially perpendicular to the first surface of the sublayer.

5. The electronic device of claim 3, further comprising:
a second electronic component disposed over the component side of the carrier,
wherein the recessed portion is between the first electronic component and the second electronic component and is configured to provide adjustment to a relative position of the first electronic component and the second electronic component.

6. The electronic device of claim 3, wherein the first protection layer further covers an interconnection structure electrically connecting the first electronic component to one of the first sensing element or the second sensing element.

7. The electronic device of claim 6, wherein the first protection layer covers a lateral surface of the interconnection structure of the carrier.

8. The electronic device of claim 1, wherein the thinned portion extends along a circumference of the first sensing element.

9. The electronic device of claim 1, wherein the thinned portion and the first sensing element are overlapped in the two directions.

10. The electronic device of claim 1, further comprising:
a third sensing element arranged along a secondary direction different from the primary direction with respect to the first sensing element, wherein the thinned portion extends between the third sensing element and the first sensing element.

11. An electronic device, comprising:
a carrier having a first surface and a second surface opposite to the first surface, wherein the carrier has a thinned portion;
a first sensing element disposed over the first surface of the carrier and adjacent to the thinned portion; and
an electronic component disposed over the second surface of the carrier,
wherein the first sensing element and the electronic component are at least partially overlapped along a direction substantially perpendicular to the second surface of the carrier.

12. The electronic device of claim 11, wherein the first sensing element is disposed on a first protruding portion of the carrier adjacent to the thinned portion.

13. The electronic device of claim 12, further comprising:
a protection layer disposed over the second surface of the carrier and having a second protruding portion covering the electronic component.

14. The electronic device of claim 13, wherein the first protruding portion and the second protruding portion are at least partially overlapped along the direction.

15. The electronic device of claim 11, wherein the first sensing element is at least partially embedded in a protruding portion of the carrier adjacent to the thinned portion.

16. An electronic device, comprising:
a carrier having a component side and a sensing side opposite to the component side, the sensing side having a thinned portion;
a first sensing element disposed over the sensing side; and
a second sensing element disposed over the sensing side,
wherein the thinned portion is between the first sensing element and the second sensing element and is configured to allow the first sensing element and the second sensing element to respectively move toward two directions, and wherein an angle between the two directions is greater than zero.

17. The electronic device of claim 16, wherein the thinned portion is configured to change a relative elevation of the first sensing element and the second sensing element.

18. The electronic device of claim 16, wherein the thinned portion is configured to allow the first sensing element and the second sensing element to face toward each other.

19. The electronic device of claim 16, wherein the carrier comprises a protruding portion over the sensing side and configured for pressing along a direction substantially perpendicular to the protruding portion.

20. The electronic device of claim 16, wherein the thinned portion is configured to provide adjustment to a relative position between the first sensing element and the second sensing element in at least two different dimensions.
